(19) United States
(12) Patent Application Publication
(10) Pub. No.: US 2024/0239770 A1
(43) Pub. Date: Jul. 18, 2024

(54) ALLYL-CONTAINING BISPHENOL RESIN

(71) Applicant: CHANG CHUN PLASTICS CO., LTD., Taipei City (TW)

(72) Inventor: Shih-De YANG, Taipei City (TW)

(21) Appl. No.: 18/401,857

(22) Filed: Jan. 2, 2024

Related U.S. Application Data
(60) Provisional application No. 63/478,288, filed on Jan. 3, 2023.

(30) Foreign Application Priority Data
Dec. 27, 2023 (TW) 112151084

(51) Int. Cl. C07D 403/10; C07D 403/12

(52) U.S. Cl. CPC C07D 403/10; C07D 403/12

(57) ABSTRACT

An allyl-containing bisphenol resin, including a first structural unit derived from a bisphenol compound containing two or more allyl groups per molecule and a polyfunctional maleimide compound. The first structural unit contains one or more allyl groups; and when the allyl-containing bisphenol resin is characterized by Fourier transform infrared spectroscopy (FTIR), the FTIR spectrum of the allyl-containing bisphenol resin has a characteristic peak A at a vibration frequency ranging from 1615 cm⁻¹ to 1765 cm⁻¹, a characteristic peak B at a vibration frequency ranging from 1615 cm⁻¹ to 1765 cm⁻¹, and a characteristic peak C at a vibration frequency ranging from 1155 cm⁻¹ to 1265 cm⁻¹. The vibration frequency of characteristic peak A is lower than peak B, the characteristic peaks A, B and C have a peak area A_{A}, peak area A_{B} and peak area A_{C}, respectively, which meet the following condition: (A_{A}/A_{C})+(A_{C}/A_{B})=1.20 to 1.60.

### CLAIM FOR PRIORITY

This application claims the benefits of U.S. Provisional Patent Application No. 63/478,288 filed on Jan. 3, 2023 and Taiwan Patent Application No. 112151084 filed on Dec. 27, 2023, the subject matters of which are incorporated herein in their entirety by reference.

### BACKGROUND OF THE INVENTION

### Field of the Invention

The present application provides an allyl-containing bisphenol resin, which has specific infrared spectrum properties and/or comprises a specific structure.

### Descriptions of the Related Art

As the applications of electronic products have gradually evolved toward lighter, thinner, shorter, smaller and more multi-functional designs, the requirements for the properties of electronic materials have increased. Particularly, a printed circuit board (PCB), serving as the primary supportive substrate for electronic components, should possess characteristics such as high-density wiring, thinness, high dimensional stability, and efficient heat dissipation.

However, heat may be generated during the processing of PCBs, particularly through high temperature generated during drilling caused by a high rotational speed of the drill bit. As a consequence, a resin system with a lower glass transition temperature (T_{g}) and coefficient of thermal expansion (CTE) is prone to softening during PCB processing. This softening can lead to the detachment of copper wire(s) and deformation of the PCBs, significantly impacting subsequent processing stages.

Furthermore, with the development of 5G systems, the requirements regarding high data transmission density and low-latency transmission have become more stringent. To prevent signal loss or delay during the transmission process caused by material defects, there is a need to enhance the resin system used in the electronic materials.

To address the aforementioned technical problems, prior art has suggested using a bismaleimide compound as part of the resin system to enhance the heat resistance, dimensional stability and electrical properties of the produced electronic material. However, bismaleimide compound has disadvantages, such as poor solubility and a short storage life, leading to restrictions in their applications. In addition, despite the resin system in the prior art employing a bismaleimide compound, the enhancements in heat resistance, processibility and dimensional stability remain insufficient.

### SUMMARY OF THE INVENTION

Considering the aforementioned technical problems, the present application aims to modify the bismaleimide compound to address the restrictions in applications due to such as poor solubility and a short storage life. By means of the modification, the present application can provide a new resin with excellent physiochemical properties. Specifically, the present application provides an allyl-containing bisphenol resin, which comprises a structural unit derived from a bisphenol compound containing two or more allyl groups per molecule and a polyfunctional maleimide compound. The cured product prepared from the allyl-containing bisphenol resin has superior heat resistance and dimensional stability, and may further has superior high temperature stability, toughness, hydrophobicity, adhesion to copper foils and/or dielectric properties, making it particularly suitable for application as a dielectric material in printed circuit boards. Additionally, the allyl-containing bisphenol resin demonstrates good stability, enabling long-term storage. Due to its exceptional properties, the allyl-containing bisphenol resin can be used in smaller quantities to achieve greater efficacy. This allows for a reduction in the amount of maleimide compound(s) used and offers advantages such as improved solubility and operability.

Therefore, an objective of the present application is to provide an allyl-containing bisphenol resin, which comprises a first structural unit derived from a bisphenol compound containing two or more allyl groups per molecule and a polyfunctional maleimide compound, wherein the first structural unit contains one or more allyl groups; and when the allyl-containing bisphenol resin is characterized by Fourier transform infrared spectroscopy (FTIR), the FTIR spectrum of the allyl-containing bisphenol resin has a characteristic peaks A at a vibration frequency ranging from 1615 cm⁻¹ to 1765 cm⁻¹, a characteristic peak B at a vibration frequency ranging from 1615 cm⁻¹ to 1765 cm⁻¹, and a characteristic peak C at a vibration frequency ranging from 1155 cm⁻¹ to 1265 cm⁻¹, wherein the vibration frequency of characteristic peak A is lower than that of characteristic peak B, and preferably, the characteristic peak A is at a vibration frequency ranging from 1615 cm⁻¹ to 1690 cm⁻¹, and the characteristic peak B is at a vibration frequency ranging from higher than 1690 cm⁻¹ to 1765 cm⁻¹. The characteristic peaks A, B and C have corresponding peak areas A_{A}, A_{B} and A_{C}, respectively, which meet the following condition: (A_{A}/A_{C})+(A_{C}/A_{B})=1.20 to 1.60, preferably (A_{A}/A_{C})+(A_{C}/A_{B})=1.25 to 1.52.

In some embodiments of the present application, A_{A}/A_{C}=0.25 to 0.60.

In some embodiments of the present application, A_{C}/A_{B}=0.70 to 1.40.

In some embodiments of the present application, the Fourier transform infrared spectroscopy is performed as follows by using a Fourier transform infrared spectrometer: coating the allyl-containing bisphenol resin onto a KBr pellet by a film method, and measuring an absorption spectrum of the coated KBr pellet over a scan range of 400 cm⁻¹ to 4000 cm⁻¹ by using a transmission type method under a resolution of 1 cm⁻¹ and a scan number of 16.

In some embodiments of the present application, the bisphenol compound containing two or more allyl groups per molecule is selected from the group consisting of a bisphenol A containing two or more allyl groups per molecule, a bisphenol B containing two or more allyl groups per molecule, a bisphenol F containing two or more allyl groups per molecule, a bisphenol S containing two or more allyl groups per molecule, a bisphenol Z containing two or more allyl groups per molecule, a 4,4′-dihydroxydiphenyl ether compound containing two or more allyl groups per molecule, and combinations thereof.

In some embodiments of the present application, the allyl-containing bisphenol resin further comprises a polyfunctional maleimide compound.

Another objective of the present application is to provide an allyl-containing bisphenol resin, which comprises a structure of Formula (I) below:

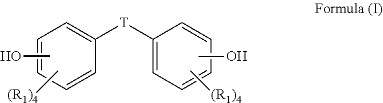

wherein,
each R₁ is independently —H, —CH₃,

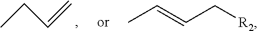

with the proviso that at least one R₁ is

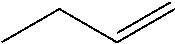

and at least one R₁ is

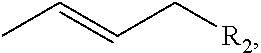

wherein R₂ is a group derived from a polyfunctional maleimide; and
T is —CH₂—, —C(CH₃)₂—, —C(CH₃)(CH₂CH₃)—, —SO₂—, —O—, or

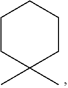

and is —CH₂— or —C(CH₃)₂— in some embodiments.

In some embodiments of the present application, the allyl-containing bisphenol resin comprises a structure of Formula (I-1) below:

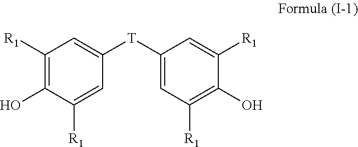

wherein at least one R₁ is

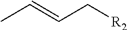

and the rest are

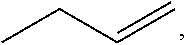

wherein R₂ is a group derived from a polyfunctional maleimide; and
T is —CH₂—, —C(CH₃)₂—, —C(CH₃)(CH₂CH₃)—, —SO₂—, —O—, or

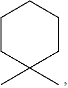

and is —CH₂— or —C(CH₃)₂— in some embodiments.

In some embodiments of the present application, R₂ is

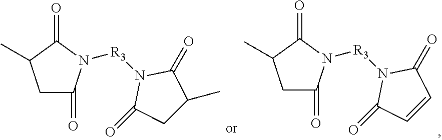

wherein R₃ is selected from the group consisting of

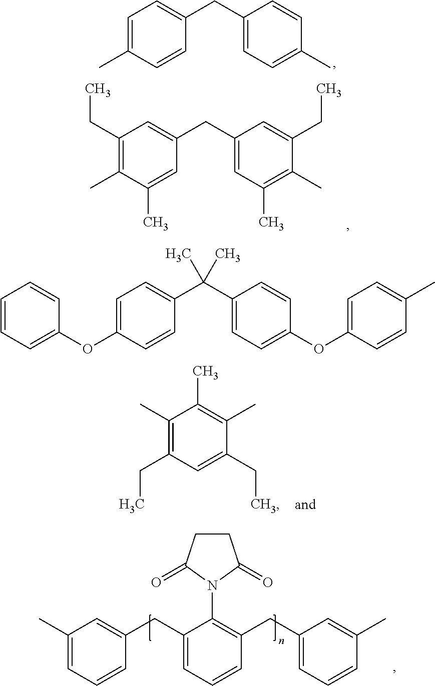

wherein n is an integer of 1 to 10.

In some embodiments of the present application, when the allyl-containing bisphenol resin is characterized by gel permeation chromatography (GPC) and an absorbance detector, the allyl-containing bisphenol resin has an absorption peak at 254 am, the absorption peak has an area A₂₅₄, and a ratio of area A₂₅₄ to the total area of all absorption peaks is 15% or less, wherein the testing conditions of the gel permeation chromatography (GPC) and the absorbance detector are as follows: diluting the allyl-containing bisphenol resin with tetrahydrofuran to a concentration of 200 ppm; feeding at a flow rate of 1.0 mL/min to a series of columns, consisting of one column C1, one column C2, and two columns C3 in the said sequence, to perform separation, wherein the column C1 has a length of 30 cm, an inner diameter of 7.8 mm and is filled with a polystyrene-divinylbenzene having an average particle size of 5 μm and an average pore size of 7.5 nm, the column C2 has a length of 30 cm, an inner diameter of 7.8 mm and is filled with a polystyrene-divinylbenzene having an average particle size of 5 μm and an average pore size of 2 nm, and the columns C3 each have a length of 30 cm, an inner diameter of 7.8 mm and are filled with a polystyrene-divinylbenzene having an average particle size of 5 μm and an average pore size of 1.5 nm; collecting a sample during an elution time between 20 min and 35 min and analyzing all absorption peaks within a range of 190 nm to 800 nm with an absorbance detector; and calculating the ratio of the area A₂₅₄ of the absorption peak at 254 nm to the total area of all absorption peaks.

To render the above objectives, technical features, and advantages of the present application more apparent, the present application will be described in detail with reference to some specific embodiments hereinafter.

### BRIEF DESCRIPTION OF THE DRAWINGS

Not applicable.

### DESCRIPTION OF THE PREFERRED EMBODIMENTS

Some specific embodiments of the present application will be described in detail. However, the present application may be embodied in various embodiments and should not be limited to the embodiments described in the specification.

Unless additionally explained, the expressions “a,” “the,” or the like recited in the specification and the claims should include both the singular and the plural forms.

Unless additionally explained, the expressions “first,” “second,” or the like recited in the specification and the claims are only used to distinguish the illustrated elements of components without special meanings. Those expressions are not used to represent any priority.

Unless otherwise specified, the term “allyl-containing bisphenol resin” recited in the specification and the claims refers to a resin which contains allyl groups in the molecule and has a bisphenol structure.

The primary advantage of the present application over prior art lies in enhancing the heat resistance and dimensional stability of the cured product derived from an allyl-containing bisphenol resin, along with improving the stability of the allyl-containing bisphenol resin. In some embodiments, the allyl-containing bisphenol resin further has superior high temperature stability, toughness, hydrophobicity, adhesion to copper foils and/or dielectric properties. This is achieved by controlling the infrared spectrum properties of the allyl-containing bisphenol resin and/or by incorporating a specific structure into the allyl-containing bisphenol resin. The allyl-containing bisphenol resin can be used in any technical fields that demand the aforementioned properties, including the preparations of coating materials, modification materials or dielectric materials for printed circuit boards, heat dissipation materials, aircraft wing materials, 3D printing materials, impregnating resin materials for glass fibers or carbon fibers, heat-resistant materials for substrates, spray coating materials, or bulletproof vest coating materials. However, the scope of the present application is not limited to these examples. Details regarding the allyl-containing bisphenol resin are provided below.

### 1. ALLYL-CONTAINING BISPHENOL RESIN HAVING SPECIFIC INFRARED SPECTRUM PROPERTIES

An objective of the present application is to provide an allyl-containing bisphenol resin, which has specific infrared spectrum properties and comprises a first structural unit derived from a bisphenol compound containing two or more allyl groups per molecule and a polyfunctional maleimide compound, wherein the first structural unit contains one or more allyl groups.

In some embodiments of the present application, with the premise that the allyl-containing bisphenol resin has the specific infrared spectrum properties, the allyl-containing bisphenol resin may further comprise a polyfunctional maleimide in a compound state in addition to a structural unit derived from a polyfunctional maleimide compound.

### 1.1. Polyfunctional Maleimide Compound

In the present application, a polyfunctional maleimide compound refers to a compound having at least two maleimide groups per molecule. Without being bound by any theory, it is believed that the presence of monomaleimides in the raw materials may result in reactants lacking groups capable of undergoing polymerization. This can lead to molecule chains with insufficient length and undesirable polymer properties. Therefore, the present application opts for bifunctional maleimides and/or polyfunctional maleimides with higher functionality. However, the present application does not exclude the potential use of monofunctional maleimides.

The type of the polyfunctional maleimide is not particularly limited. Examples of the polyfunctional maleimide include but are not limited to a polyfunctional maleimide compound having the structure of

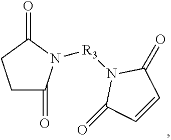

wherein R₃ can be selected from the group consisting of

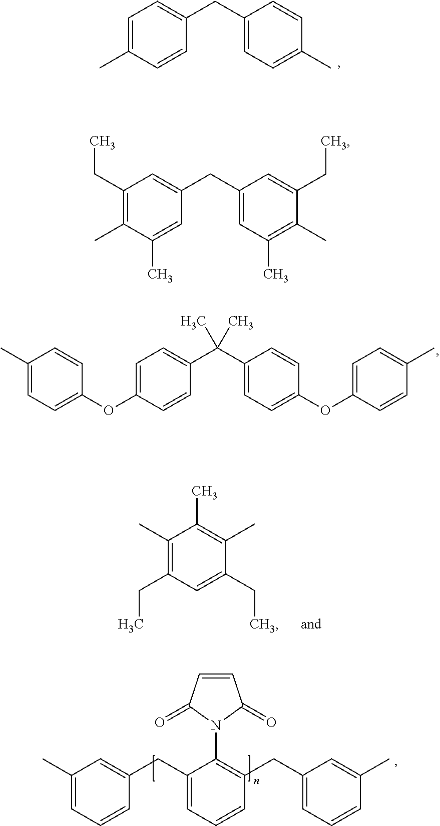

wherein n is an integer of 1 to 10. The aforementioned polyfunctional maleimides can be used alone or in any combination.

### 1.2. BISPHENOL COMPOUND CONTAINING TWO OR MORE ALLYL GROUPS PER MOLECULE

In the present application, a bisphenol compound containing two or more allyl groups per molecule denotes a compound having at least two allyls, preferably four allyls on the benzene ring(s) of its bisphenol structure. Preferably, these allyls are located at the ortho-positions relative to the hydroxyl groups.

The bisphenol compound containing two or more allyl groups per molecule can be obtained by subjecting a bisphenol to allylation. A specific preparation method is described in detail in the Examples. Examples of the bisphenol include, but are not limited to, bisphenol A, bisphenol B, bisphenol F, bisphenol S, bisphenol Z, and 4,4′-dihydroxydiphenyl ether. Therefore, examples of the bisphenol compound containing two or more allyl groups per molecule include a bisphenol A containing two or more allyl groups per molecule, a bisphenol B containing two or more allyl groups per molecule, a bisphenol F containing two or more allyl groups per molecule, a bisphenol S containing two or more allyl groups per molecule, a bisphenol Z containing two or more allyl groups per molecule, and a 4,4′-dihydroxydiphenyl ether compound containing two or more allyl groups per molecule. The aforementioned bisphenol compounds can be used alone or in any combination.

### 1.3. Infrared Spectrum Properties of Allyl Containing Bisphenol Resin

When the allyl-containing bisphenol resin is characterized by Fourier transform infrared spectroscopy (FTIR), the FTIR spectrum of the allyl-containing bisphenol resin has a characteristic peak A at a vibration frequency ranging from 1615 cm⁻¹ to 1765 cm⁻¹, a characteristic peak B at a vibration frequency ranging from 1615 cm⁻¹ to 1765 cm⁻¹, and a characteristic peak C at a vibration frequency ranging from 1155 cm⁻¹ to 1265 cm⁻¹. The vibration frequency of characteristic peak A is lower than that of characteristic peak B. Preferably, the characteristic peak A is at a vibration frequency ranging from 1615 cm⁻¹ to 1690 cm⁻¹, and the characteristic peak B is at a vibration frequency ranging from higher than 1690 cm⁻¹ to 1765 cm⁻¹.

Characteristic peaks A, B and C have peak areas A_{A}, A_{B} and A_{C}, respectively, which meet the following condition: (A_{A}/A_{C})+(A_{C}/A_{B})=1.20 to 1.60, preferably (A_{A}/A_{C})+(A_{C}/A_{B})=1.25 to 1.52. For example, (A_{A}/A_{C})+(A_{C}/A_{B}) can be 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, or 1.60, or within a range between any two of the values described herein. If the value of (A_{A}/A_{C})+(A_{C}/A_{B}) is outside the aforementioned range, the allyl-containing bisphenol resin is incapable of simultaneously providing excellent heat resistance, dimensional stability, storage properties, and stability.

With the premise that the aforementioned condition of (A_{A}/A_{C})+(A_{C}/A_{B}) is satisfied, peak area A_{A} and peak area A_{C} can further meet the following condition: A_{A}/A_{C}=0.25 to 0.60. For example. A_{A}/A_{C} can be 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, or 0.60, or within a range between any two of the values described herein.

With the premise that the aforementioned condition of (A_{A}/A_{C})+(A_{C}/A_{B}) is satisfied, peak area A_{C} and peak area A_{B} can further meet the following condition: A_{C}/A_{B}=0.70 to 1.40, preferably A_{C}/A_{B}=0.75 to 1.40, more preferably A_{C}/A_{B}=0.80 to 1.40. For example, A_{C}/A_{B} can be 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, or 1.40, or within a range between any two of the values described herein. When the A_{C}/A_{B} value falls within the aforementioned range, the resin exhibits a longer gel time, making it suitable for applications requiring an extended gel time.

The infrared spectrum properties of the allyl-containing bisphenol resin can be obtained by performing Fourier transform infrared spectroscopy as follows by using a Fourier transform infrared spectrometer: coating the allyl-containing bisphenol resin onto a KBr pellet by a film method, and measuring an absorption spectrum of the coated KBr pellet over a scan range of 400 cm⁻¹ to 4000 cm⁻¹ by using a transmission type method under a resolution of 1 cm⁻¹ and a scan number of 16. Peak areas A_{A}, A_{B} and A_{C} of the characteristic peaks A, B and C are determined by calculating the integral area defined by the line connecting the start and end points of the absorption peak within specified ranges. For example, peak area A_{A} of the characteristic peak A is obtained by calculating the integral area defined by the line connecting the start and end points of the absorption peak at 1615 cm⁻¹ to 1690 cm⁻¹; peak area A_{B} of the characteristic peak B is obtained by calculating the integral area defined by the line connecting the start and end points of the absorption peak at higher than 1690 cm⁻¹ to 1765 cm⁻¹; and peak area A_{C} of the characteristic peak C is obtained by calculating the integral area defined by the line connecting the start and end points of the absorption peak at 1155 cm⁻¹ to 1265 cm⁻¹. Specifically, a software can be used to identify points where the first derivative equals zero as the start, top, and end points of a characteristic peak. Subsequently, the software calculates the integral area defined between the start, top, and end points, providing the area measurement of the characteristic peak.

The infrared spectrum properties of the allyl-containing bisphenol resin can be controlled by adjusting the raw materials used in the preparation of the allyl-containing bisphenol resin (such as the species or equivalent weights of the raw materials) or by adjusting the process conditions of the allyl-containing bisphenol resin (such as the reaction temperature of reaction time). Persons having ordinary skill in the art can prepare an allyl-containing bisphenol resin with the aforementioned infrared spectrum properties by referring to the specification of the subject application, particularly relying on the specific illustrations in the Examples.

### 1.4. First Structural Unit

In some embodiments of the present application, the first structural unit of the allyl-containing bisphenol resin has a structure of Formula (I) below:

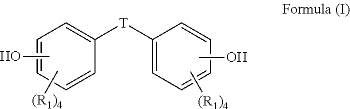

wherein,
each R₁ is independently —H, —CH₃,

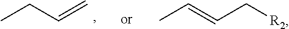

with the proviso that at least one R₁ is

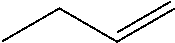

and at least one R₁ is

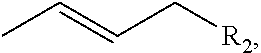

wherein R₂ is a group derived from a polyfunctional maleimide; and
T is —CH₂—, —C(CH₃)₂—, —C(CH₃)(CH₂CH₃)—, —SO₂—, —O—, or

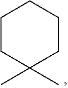

and is —CH₂— or —C(CH₃)₂— in some embodiments.

More specifically, the first structural unit of the allyl-containing bisphenol resin may have a structure of Formula (I-1) below:

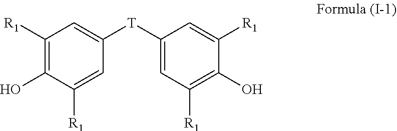

wherein at least one R₁ is

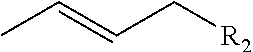

and the rest are

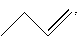

wherein R₂ is a group derived from a polyfunctional maleimide; and
T is —CH₂—, —C(CH₃)₂—, —C(CH₃)(CH₂CH₃)—, —SO₂—, —O—, or

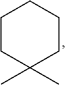

and is —CH₂— or —C(CH₃)₂— in some embodiments.

Preferably, in the structures of Formula (I) and Formula (I-1), R₂ is preferably derived from a polyfunctional maleimide having a structure of

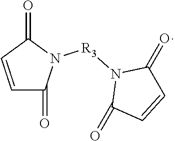

Therefore R₂ is preferably

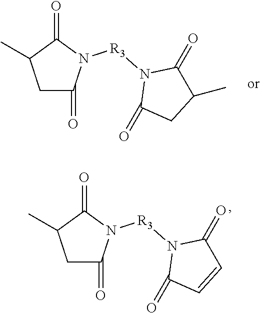

wherein R₃ is selected from the group consisting of

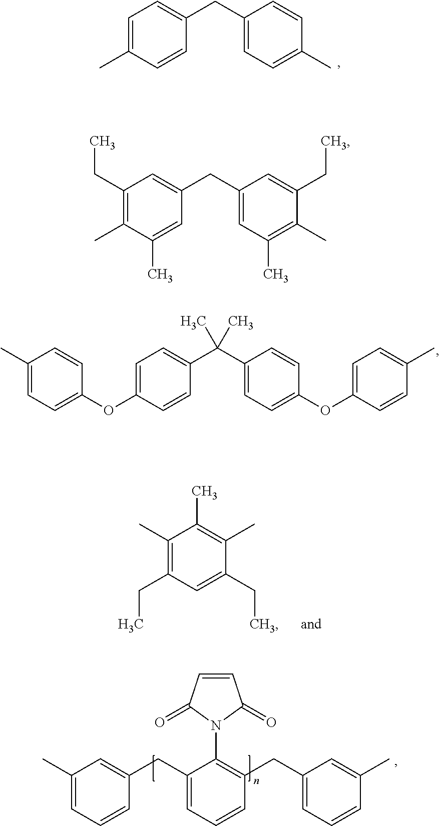

wherein n is an integer of 1 to 10.

In some embodiments of the present application, T in formula (I) and formula (I-1) is —SO₂—, and the cured product of the allyl-containing bisphenol resin has further improved high temperature stability and toughness.

In some embodiments of the present application, T in formula (I) and formula (I-1) is —O—, and the cured product of the allyl-containing bisphenol resin has further improved hydrophobicity and adhesion to copper foils.

In some embodiments of the present application, T in formula (I) and formula (I-1) is

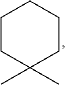

and the cured product of the allyl-containing bisphenol resin has further improved dielectric properties.

### 1.5. Preparation of Allyl-Containing Bisphenol Resin

The allyl-containing bisphenol resin can be prepared through a cross-linking reaction involving a polyfunctional maleimide compound and a bisphenol compound containing two or more allyl groups per molecule, such as a bisphenol compound containing four allyl groups per molecule. The FTIR properties of the allyl-containing bisphenol resin can be controlled by the selection of a bisphenol compound containing two or more allyl groups per molecule, and by adjusting the reaction temperature or reaction time of the polyfunctional maleimide compound and the bisphenol compound containing two or more allyl groups per molecule. For example, the allyl-containing bisphenol resin can be prepared by using a bisphenol compound containing four allyl groups per molecule, and performing reaction at a reaction temperature of 90° C. to 130° C. for 90 minutes to 500 minutes.

### 2. ALLYL-CONTAINING BISPHENOL RESIN COMPRISING SPECIFIC STRUCTURE

Another objective of the present application is to provide an allyl-containing bisphenol resin, which comprises a structure of Formula (I) below:

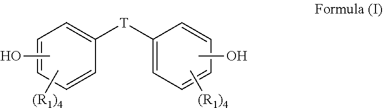

wherein
each R₁ is independently —H, —CH₃;

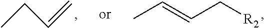

with the proviso that at least one R₁ is

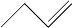

and at least one R₁ is

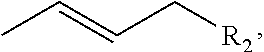

wherein R₂ is a group derived from a polyfunctional maleimide; and
T is —CH₂—, —C(CH₃)₂—, —C(CH₃)(CH₂CH₃)—, —SO₂—, —O—, or

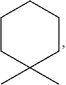

and is —CH₂— or —C(CH₃)₂— in some embodiments.

In some embodiments of the present application, the allyl-containing bisphenol resin comprises a structure of Formula (I-1) below:

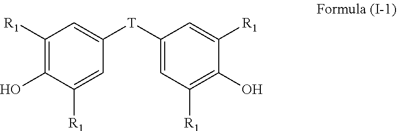

wherein at least one R₁ is

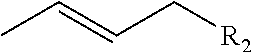

and the rest are

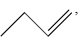

wherein R₂ is a group derived from a polyfunctional maleimide; and
T is —CH₂—, —C(CH₃)₂—, —C(CH₃)(CH₂CH₃)—, —SO₂—, —O—, or

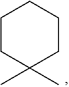

and is —CH₂— or —C(CH₃)₂— in some embodiments.

Preferably, in the structures of Formula (I) and Formula (1-1), R₂ is preferably derived from a polyfunctional maleimide having a structure of

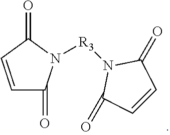

Therefore, R₂ is preferably

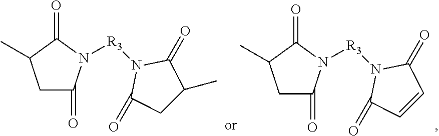

wherein R₃ is selected from the group consisting of

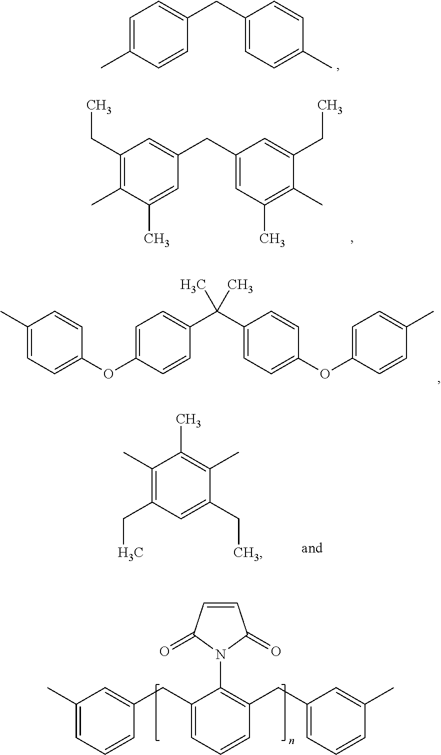

wherein n is an integer of 1 to 10.

The allyl-containing bisphenol resin can be prepared by subjecting a polyfunctional maleimide compound and a bisphenol compound containing two or more allyl groups per molecule, such as a bisphenol compound containing four allyl groups per molecule, to a cross-linking reaction. The bisphenol compound containing two or more allyl groups per molecule can be prepared by subjecting a bisphenol to allylation. Specific preparation methods are illustrated in the Examples.

In some embodiments of the present application, when the allyl-containing bisphenol resin is characterized by gel permeation chromatography (GPC) and an absorbance detector, the allyl-containing bisphenol resin has an absorption peak at 254 nm, the absorption peak has an area A₂₅₄, and the ratio of area A₂₅₄ to the total area of all absorption peaks is 15% or less. The testing conditions of the gel permeation chromatography (GPC) and the absorbance detector are as follows: diluting the allyl-containing bisphenol resin with tetrahydrofuran to a concentration of 200 ppm; feeding at a flow rate of 1.0 mL/min to a series of gel permeation chromatography columns in sequence to perform separation; collecting a sample with an elution time ranging from 20 min to 35 min and analyzing all absorption peaks within a range of 190 nm to 800 nm with an absorbance detector; and calculating ratio of the area A₂₅₄ of the absorption peak at 254 nm to the total area of all absorption peaks. In the present application, the aforementioned columns is a series of column consisting of one column C1, one column C2, and two columns C3 in the said sequence, wherein the column C1 has a length of 30 cm, an inner diameter of 7.8 mm and is filled with a polystyrene-divinylbenzene having an average particle size of 5 μm and an average pore size of 7.5 nm, such as a TSK gel G3000Hxl product; the column C2 has a length of 30 cm, an inner diameter of 7.8 mm and is filled with a polystyrene-divinylbenzene having an average particle size of 5 μm and an average pore size of 2 nm, such as a TSK gel G2000Hxl product; and the columns C3 each have a length of 30 cm, an inner diameter of 7.8 mm and are filled with a polystyrene-divinylbenzene having an average particle size of 5 μm and an average pore size of 1.5 nm, such as a TSK gel G1000Hxl product. In the present application, a software can be used to identify points where the first derivative equals zero as the start, top, and end points of a absorption peak. Subsequently, the software calculates the integral area defined between the start, top, and end points, providing the area measurement of the absorption peak.

### 3. EXAMPLES

### 3.1. Testing Methods

The present application is further illustrated by the embodiments hereinafter. wherein the testing instruments and methods are as follows.

### [Infrared Spectroscopy Test]

1 mg of the synthesized allyl-containing bisphenol resin is coated onto a one-millimeter thick KBr pellet by a film method. The coated KBr pellet is placed on the pellet holder and inserted into the Fourier transform infrared spectrometer. The absorption spectrum of the coated KBr pellet is measured over a scan range of 400 cm⁻¹ to 4000 cm^{31 1} by using a transmission type method under a resolution of 1 cm⁻¹ and a scan number of 16. The peak areas A_{A}, A_{B} A_{C} are determined by calculating the integral area defined by the line connecting the start and end points of the absorption peak located within a given range using PerkinElmer@ Spectrum version 10.5.3 software.

### [Glass Transition Temperature (T_{g}) Test]

The prepared resin is placed in a high-temperature oven. It is maintained at 180° C. under a vacuum condition for 1 hour to remove solvent. Subsequently, it undergoes a pre-cross-linking at 200° C. for 2 hours and proceeds to complete curing at 250° C. for 6 hours. The cured product is cut into dimensions of 10 mm×10 mm×5 mm. The cut products are then subjected to analysis using a thermal mechanical analyzer (TMA, model number: Waters Q400). The test conditions are: raising the temperature with a heating rate of 10° C./min from 30° C. to 330° C. and then reducing the temperature with a cooling rate of 10° C./min from 330° C. to 30° C. to complete calibration; and then raising the temperature with a heating rate of 10° C./min from 30° C. to 330° C. with the glass transition temperature (T_{g}) being recorded.

### [Thermogravimetric Analysis Test (TGA Test)]

The prepared resin is placed in a high-temperature oven. It is maintained at 180° C. under a vacuum condition for 1 hour to remove solvent. Subsequently, it undergoes a pre-cross-linking at 200° C. for 2 hours and proceeds to complete curing at 250° C. for 6 hours. 10 mg of the cured product is subjected to analysis using a thermogravimetric analyzer (TGA, model number: Waters Q500), wherein the temperature conditions are as follows: balancing at 50° C. for 5 minutes and then raising to 800° C. with a rate of 10° C./min. The temperature at which the weight loss percentage of the thermally cured product reaches 5wt % (T_{d5}) is recorded.

### [Thermal Coefficient of Expansion (CTE) Test]

The prepared resin is placed in a high-temperature oven. It is maintained at 180° C. under a vacuum condition for 1 hour to remove solvent. Subsequently, it undergoes a pre-cross-linking at 200° C. for 2 hours and proceeds to complete curing at 250° C. for 6 hours. The cured product is cut into dimensions of 10 mm×10 mm×5 mm. The cut products are then subjected to analysis using a thermal mechanical analyzer (TMA, model number: Waters Q400). The test conditions are: raising the temperature with a heating rate of 10° C./min from 30° C. to 330° C. and then reducing the temperature with a cooling rate of 10° C./min from 330° C. to 30° C. to complete calibration; and then raising the temperature with a heating rate of 10° C./min from 30° C. to 330° C., with the thermal coefficient of expansion (CTE) from 50° C. to 260° C. being recorded.

### [GPC Test]

The prepared resin is diluted with tetrahydrofuran to 200 ppm and then placed into an autosampler (model number: Waters 717 plus Autosampler). The sample is fed by a pump (model number: Waters 515 pump) with a flow rate of 1.0 mL/min to a series of gel permeation chromatography columns in sequence (model number and amount: TSK gel G3000HXL*1+G2000HXL*1+G1000HXL*2) for separation and the sample eluted from 20 min to 35 min is collected. An absorbance detector (model number: Waters 2487 Dual λ Absorbance Detector) is used to analyze all absorption peaks within a range of 190 nm to 800 nm. After that, a chromatography analysis software SISC32-GPC available from Scientific Information Service Company is used to calculate the ratio of the area A₂₅₄ of the absorption peak at 254 nm to the total area of all absorption peaks.

### [Precipitation Test]

The prepared resin is placed in a high-temperature oven. It is maintained at 180° C. under a vacuum condition for 1 hour to remove solvent. Subsequently, it undergoes a pre-cross-linking at 200° C. for 2 hours to obtain a pre-cross-linked product. The pre-cross-linked product is dissolved in a mixed solvent of 1:1 methyl ethyl ketone (MEK) and propylene glycol methyl ether (PGME), forming a solution with a solid content of 60 wt % to 75 wt %. The obtained solution is left at 30° C. for 3 days and observed for any precipitate, which indicates the formation of phase heterogeneity.

### 3.2. Synthesis of Bisphenol Compound Containing Two or More Allyl Groups Per Molecule

### Synthesis Example 1

228 g of bisphenol A (available from Chang Chun Plastics Company, CAS number: 80-05-7) was dissolved in dimethyl sulfoxide (CAS number: 67-68-5). Subsequently, 170 g of 50% NaOH (CAS number: 1310-73-2) was added and stirred under room temperature until a clear solution was obtained. The obtained clear solution was then heated to 70° C., and 170 g of allyl chloride (CAS number: 107-05-1) was added until the reaction was complete. The reaction product was heated to 200° C. under a vacuum condition and stirred for 2 hours at 200° C. to yield an intermediate product.

The intermediate product was dissolved in dimethyl sulfoxide (CAS number: 67-68-5). Subsequently, 170 g of 50% NaOH (CAS number: 1310-73-2) was added and stirred under room temperature until a clear solution was obtained. The obtained clear solution was then heated to 70° C., and 170 g of allyl chloride (CAS number: 107-05-1) was added until the reaction was complete. The reaction product was heated to 200° C. under a vacuum condition and stirred for 2 hours at 200° C. to yield a bisphenol A containing four allyls.

### 3.3. Preparation of Allyl-Containing Bisphenol Resin

### Example 1

58.2 g of the bisphenol A containing four allyls, synthesized in Synthesis Example 1, and 53.7 g of BMI-1000 (a bismaleimide available from DKK Company) were added to a 200 mL reactor. The mixture was stirred at a rotational speed of 200 rpm and 130° C. for 2 hours to initiate the reaction, resulting in the allyl-containing bisphenol resin E1 of Example 1.

### Example 2

58.2 g of the bisphenol A containing four allyls, synthesized in Synthesis Example 1, and 55.8 g of BES1-5950 (a polyfunctional maleimide available from Regina Company) were added to a 200 mL reactor. The mixture was stirred at a rotational speed of 200 rpm and 130° C. for 2 hours to initiate the reaction, resulting in the allyl-containing bisphenol resin E2 of Example 2.

### Example 3

58.2 g of the bisphenol A containing four allyls, synthesized in Synthesis Example 1, and 66.4 g of BMI-70 (a bismaleimide available from KI Chemical Company) were added to a 200 mL reactor. The mixture was stirred at a rotational speed of 200 rpm and 130° C. for 2 hours to initiate the reaction, resulting in the allyl-containing bisphenol resin E3 of Example 3.

### Example 4

58.2 g of the bisphenol A containing four allyls, synthesized in Synthesis Example 1, and 42.3 g of TDAB (a bismaleimide available from EVONIK Company) were added to a 200 mL reactor. The mixture was stirred at a rotational speed of 200 rpm and 130° C. for 2 hours to initiate the reaction, resulting in the allyl-containing bisphenol resin E4 of Example 4.

### Example 5

58.2 g of the bisphenol A containing four allyls, synthesized in Synthesis Example 1, and 53.7 g of BMI-1000 (a bismaleimide available from DKK Company) were added to a 200 mL reactor. The mixture was stirred at a rotational speed of 100 rpm and 130° C. for 2 hours to initiate the reaction, resulting in the allyl-containing bisphenol resin E5 of Example 5.

### Example 6

58.2 g of the bisphenol A containing four allyls, synthesized in Synthesis Example 1, and 55.8 g of BES1-5950 (a polyfunctional maleimide available from Regina Company) were added to a 200 mL reactor. The mixture was stirred at a rotational speed of 100 rpm and 130° C. for 2 hours to initiate the reaction, resulting in the allyl-containing bisphenol resin E6 of Example 6.

### Comparative Example 1

100 g of 8292N75 (a maleimide resin available from Huntsman Company) is taken as the resin CE1 of Comparative Example 1.

### Comparative Example 2

46.2 g of a bisphenol A containing two allyls (CAS number: 1745-89-7) and 53.7 g of Homide-121 (a bismaleimide available from HOS Company) were added to a 200 mL reactor. The mixture was stirred at a rotational speed of 200 rpm and 130° C. for 2 hours to initiate the reaction, resulting in the allyl-containing bisphenol resin CE2 of Comparative Example 2.

### Comparative Example 3

46.2 g of a bisphenol containing two allyls (CAS number: 1745-89-7) and 53.7 g of BMI-1000 (a bismaleimide available from DKK Company) were added to a 200 mL reactor. The mixture was stirred at a rotational speed of 200 rpm and 130° C. for 2 hours to initiate the reaction, resulting in the allyl-containing bisphenol resin CE3 of Comparative Example 3.

### Comparative Example 4

58.2 g of the bisphenol A containing four allyls, synthesized in Synthesis Example 1, and 53.7 g of BMI-1000 (a bismaleimide available from DKK Company) were added to a 200 mL reactor. The mixture was stirred at a rotational speed of 200 rpm and 130° C. for 0.5 hours to initiate the reaction, resulting in the allyl-containing bisphenol resin CE4 of Comparative Example 4.

### Comparative Example 5

58.2 g of the bisphenol A containing four allyls, synthesized in Synthesis Example 1, and 53.7 g of BMI-1000 (a bismaleimide available from DKK Company) were added to a 200 mL reactor. The mixture was stirred at a rotational speed of 200 rpm and 130° C. for 1 hour to initiate the reaction, resulting in the allyl-containing bisphenol resin CE5 of Comparative Example 5.

### Comparative Example 6

58.2 g of the bisphenol A containing four allyls, synthesized in Synthesis Example 1, and 55.8 g of BES1-5950 (a polyfunctional maleimide available from Regina Company) were added to a 200 mL reactor. The mixture was stirred at a rotational speed of 200 rpm and 130° C. for 0.5 hours to initiate the reaction, resulting in the allyl-containing bisphenol resin CE6 of Comparative Example 6.

### Comparative Example 7

58.2 g of the bisphenol A containing four allyls, synthesized in Synthesis Example 1, and 55.8 g of BES1-5950 (a polyfunctional maleimide available from Regina Company) were added to a 200 mL reactor. The mixture was stirred at a rotational speed of 200 rpm and 130° C. for 1 hour to initiate the reaction, resulting in the allyl-containing bisphenol resin CE7 of Comparative Example 7.

### 3.4. Property Test of Allyl-Containing Bisphenol Resin

The properties of the resin of Examples E1 to E6 and Comparative Examples CE1 to CE7, including infrared spectrum properties, glass transition temperature, thermogravimetric analysis property, coefficient of thermal expansion, GPC performance, and precipitation property were tested according to the aforementioned testing methods, and the results are tabulated in the following Table 1-1 and Table 1-2.

TABLE 1-1

| | | E1 | E2 | E3 | E4 | E5 | E6 |
|---|---|---|---|---|---|---|---|
| Infrared | A_{A}/A_{C} + A_{C}/A_{B} | 1.3 | 1.52 | 1.32 | 1.25 | 1.33 | 1.27 |
| spectrum | A_{A}/A_{C} | 0.35 | 0.27 | 0.33 | 0.36 | 0.58 | 0.36 |
| properties | A_{C}/A_{B} | 0.95 | 1.25 | 0.99 | 0.89 | 0.75 | 0.91 |

| | | | | | | |
|---|---|---|---|---|---|---|
| T_{g} | 279 | 281 | 277 | 272 | 279 | 281 |
| T_{d5} | 424 | 425 | 411 | 417 | 424 | 425 |
| CTE | 1.57 | 1.66 | 1.73 | 1.55 | 1.57 | 1.66 |
| GPC A₂₅₄ % | 8% | 11% | 15% | 15% | 11% | 8% |
| Precipitation | No | No | No | No | No | No |

TABLE 1-2

| | | CE1 | CE2 | CE3 | CE4 | CE5 | CE6 | CE7 |
|---|---|---|---|---|---|---|---|---|
| Infrared | A_{A}/A_{C} + A_{C}/A_{B} | 0.92 | 0.93 | 0.94 | 1.04 | 0.97 | 0.85 | 0.99 |
| spectrum | A_{A}/A_{C} | 0.26 | 0.16 | 0.24 | 0.70 | 0.66 | 0.48 | 0.41 |
| properties | A_{C}/A_{B} | 0.66 | 0.77 | 0.7 | 0.34 | 0.31 | 0.39 | 0.58 |

| | | | | | | | |
|---|---|---|---|---|---|---|---|
| T_{g} | 280 | 311 | 275 | 279 | 279 | 281 | 281 |
| T_{d5} | 371 | 388 | 381 | 424 | 424 | 425 | 425 |
| CTE | 2.18 | 2.06 | 2.11 | 1.57 | 1.57 | 1.66 | 1.66 |
| GPC A₂₅₄ % | 9% | 11% | 12% | 39% | 26% | 29% | 18% |
| Precipitation | No | No | No | Yes | Yes | Yes | Yes |

As shown in Table 1-1 and Table 1-2, the cured products of the allyl-containing bisphenol resins of Examples E1 to E6 of the present application have superior heat resistance and dimensional stability, and precipitation does not occur from the allyl-containing bisphenol resins, indicating good stability. By contrast, Comparative Examples CE1 to CE7 which are not the allyl-containing bisphenol resin of the present application cannot simultaneously achieve superior beat resistance, dimensional stability, and stability.

The above examples illustrate the principle and efficacy of the present application and show the inventive features thereof. People skilled in this field may proceed with various modifications and replacements based on the disclosures and suggestions of the invention as described without departing from the principle thereof. Therefore, the scope of protection of the present application is as defined in the claims as appended.

1. An allyl-containing bisphenol resin, which comprises a first structural unit derived from a bisphenol compound containing two or more allyl groups per molecule and a polyfunctional maleimide compound, wherein the first structural unit contains one or more allyl groups; and when the allyl-containing bisphenol resin is characterized by Fourier transform infrared spectroscopy (FTIR), the FTIR spectrum of the allyl-containing bisphenol resin has a characteristic peak A at a vibration frequency ranging from 1615 cm⁻¹ to 1765 cm⁻¹, a characteristic peak B at a vibration frequency ranging from 1615 cm⁻¹ to 1765 cm⁻¹, and a characteristic peak C at a vibration frequency ranging from 1155 cm⁻¹ to 1265 cm⁻¹, wherein the vibration frequency of characteristic peak A is lower than that of characteristic peak B, and the characteristic peaks A, B and C have corresponding peak areas A_{A}, A_{B} and A_{C}, respectively, which meet the following condition: (A_{A}/A_{C})+(A_{C}/A_{B})=1.20 to 1.60.

2. The allyl-containing bisphenol resin of claim 1, wherein the characteristic peak A is at a vibration frequency ranging from 1615 cm⁻¹ to 1690 cm⁻¹, and the characteristic peak B is at a vibration frequency ranging from higher than 1690 cm⁻¹ to 1765 cm⁻¹.

3. The allyl-containing bisphenol resin of claim 1, wherein the peak area A_{A}, peak area A_{B} and peak area A_{C} meet the following condition: (A_{A}/A_{C})+(A_{C}/A_{B})=1.25 to 1.52.

4. The allyl-containing bisphenol resin of claim 1, wherein A_{A}/A_{C}=0.25 to 0.60.

5. The allyl-containing bisphenol resin of claim 1, wherein A_{C}/A_{B}=0.70 to 1.40.

6. The allyl-containing bisphenol resin of claim 1, wherein the Fourier transform infrared spectroscopy is performed as follows by using a Fourier transform infrared spectrometer: coating the allyl-containing bisphenol resin onto a KBr pellet by a film method, and measuring an absorption spectrum of the coated KBr pellet over a scan range of 400 cm⁻¹ to 4000 cm⁻¹ by using a transmission type method under a resolution of 1 cm⁻¹ and a scan number of 16.

7. The allyl-containing bisphenol resin of claim 1, wherein the bisphenol compound containing two or more allyl groups per molecule is selected from the group consisting of a bisphenol A containing two or more allyl groups per molecule, a bisphenol B containing two or more allyl groups per molecule, a bisphenol F containing two or more allyl groups per molecule, a bisphenol S containing two or more allyl groups per molecule, a bisphenol Z containing two or more allyl groups per molecule, a 4,4′-dihydroxydiphenyl ether compound containing two or more allyl groups per molecule, and combinations thereof.

8. The allyl-containing bisphenol resin of claim 2, wherein the bisphenol compound containing two or more allyl groups per molecule is selected from the group consisting of a bisphenol A containing two or more allyl groups per molecule, a bisphenol B containing two or more allyl groups per molecule, a bisphenol F containing two or more allyl groups per molecule, a bisphenol S containing two or more allyl groups per molecule, a bisphenol Z containing two or more allyl groups per molecule, a 4,4′-dihydroxydiphenyl ether compound containing two or more allyl groups per molecule, and combinations thereof.

9. The allyl-containing bisphenol resin of claim 3, wherein the bisphenol compound containing two or more allyl groups per molecule is selected from the group consisting of a bisphenol A containing two or more allyl groups per molecule, a bisphenol B containing two or more allyl groups per molecule, a bisphenol F containing two or more allyl groups per molecule, a bisphenol S containing two or more allyl groups per molecule, a bisphenol Z containing two or more allyl groups per molecule, a 4,4′-dihydroxydiphenyl ether compound containing two or more allyl groups per molecule, and combinations thereof.

10. The allyl-containing bisphenol resin of claim 4, wherein the bisphenol compound containing two or more allyl groups per molecule is selected from the group consisting of a bisphenol A containing two or more allyl groups per molecule, a bisphenol B containing two or more allyl groups per molecule, a bisphenol F containing two or more allyl groups per molecule, a bisphenol S containing two or more allyl groups per molecule, a bisphenol Z containing two or more allyl groups per molecule, a 4,4′-dihydroxydiphenyl ether compound containing two or more allyl groups per molecule, and combinations thereof.

11. The allyl-containing bisphenol resin of claim 5, wherein the bisphenol compound containing two or more allyl groups per molecule is selected from the group consisting of a bisphenol A containing two or more allyl groups per molecule, a bisphenol B containing two or more allyl groups per molecule, a bisphenol F containing two or more allyl groups per molecule, a bisphenol S containing two or more allyl groups per molecule, a bisphenol Z containing two or more allyl groups per molecule, a 4,4′-dihydroxydiphenyl ether compound containing two or more allyl groups per molecule, and combinations thereof.

12. The allyl-containing bisphenol resin of claim 1, further comprising a polyfunctional maleimide compound.

13. The allyl-containing bisphenol resin of claim 2, further comprising a polyfunctional maleimide compound.

14. The allyl-containing bisphenol resin of claim 3, further comprising a polyfunctional maleimide compound.

15. The allyl-containing bisphenol resin of claim 4, further comprising a polyfunctional maleimide compound.

16. The allyl-containing bisphenol resin of claim 5, further comprising a polyfunctional maleimide compound.

17. An allyl-containing bisphenol resin, which comprises a structure of Formula (I) below: 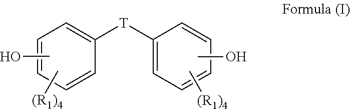
wherein, each R₁ is independently —H, —CH₃, 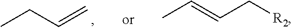
with the proviso that at least one R₁ is 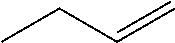
and at least one R₁ is 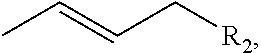
wherein R₂ is a group derived from a polyfunctional maleimide; and
T is —CH₂—, —C(CH₃)₂—, —C(CH₃)(CH₂CH₃)—, —SO₂—, —O—, or

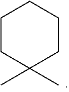

18. The allyl-containing bisphenol resin of claim 17, which comprises a structure of Formula (I-1) below: 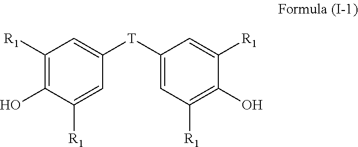
wherein at least one R₁ is 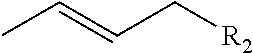
and the rest are 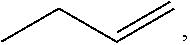
wherein R₂ is a group derived from a polyfunctional maleimide; and
T is —CH₂—, —C(CH₃)₂—, —C(CH₃)(CH₂CH₃)—, —SO₂—, —O—, or

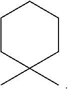

19. The allyl-containing bisphenol resin of claim 17, wherein R₂ is 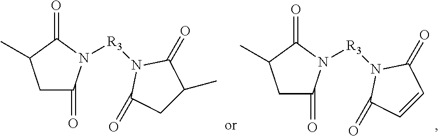
wherein R₃ is selected from the group consisting of 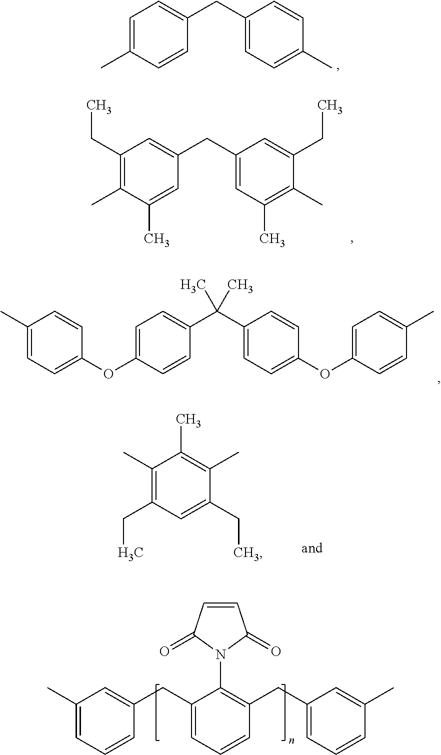
wherein n is an integer of 1 to 10.

20. The allyl-containing bisphenol resin of claim 17, wherein when the allyl-containing bisphenol resin is characterized by gel permeation chromatography (GPC) and an absorbance detector, the allyl-containing bisphenol resin has an absorption peak at 254 nm, the absorption peak has an area A₂₅₄, and a ratio of area A₂₅₄ to the total area of all absorption peaks is 15% or less, wherein the testing conditions of the gel permeation chromatography (GPC) and the absorbance detector are as follows: diluting the allyl-containing bisphenol resin with tetrahydrofuran to a concentration of 200 ppm; feeding at a flow rate of 1.0 mL/min to a series of columns, consisting of one column C1, one column C2, and two columns C3 in the said sequence, to perform separation, wherein the column C1 has a length of 30 cm, an inner diameter of 7.8 mm and is filled with a polystyrene-divinylbenzene having an average particle size of 5 μm and an average pore size of 7.5 nm, the column C2 has a length of 30 cm, an inner diameter of 7.8 mm and is filled with a polystyrene-divinylbenzene having an average particle size of 5 μm and an average pore size of 2 nm, and the columns C3 each have a length of 30 cm, an inner diameter of 7.8 mm and are filled with a polystyrene-divinylbenzene having an average particle size of 5 μm and an average pore size of 1.5 nm; collecting a sample during an elution time between 20 min and 35 min and analyzing all absorption peaks within a range of 190 nm to 800 nm with an absorbance detector; and calculating the ratio of the area A₂₅₄ of the absorption peak at 254 nm to the total area of all absorption peaks.
